(19) United States
(12) Patent Application Publication
(10) Pub. No.: US 2026/0341478 A1
(43) Pub. Date: Oct. 29, 2026

(54) PREDICTION OF TREATMENT RESPONSE IN DIABETIC MACULAR EDEMA PATIENTS

(71) Applicant: Genentech, Inc., South San Francisco, CA (US)

(72) Inventors: Matthew Kornelius MCLEOD, Sunnyvale, CA (US); Yusuke Alexander KIKUCHI, South San Francisco, CA (US); Daniela Ferrara CAVALCANTI, Marlborough, MA (US); Qi YANG, Foster City, CA (US); Neha Sutheekshna ANEGONDI, Fremont, CA (US)

(21) Appl. No.: 19/699,866

(22) Filed: Jun. 5, 2026

Related U.S. Application Data
(63) Continuation of application No. PCT/US2024/059200, filed on Dec. 9, 2024.
(60) Provisional application No. 63/608,000, filed on Dec. 8, 2023.
(60) Provisional application No. 63/642,067, filed on May 3, 2024.

(51) Int. Cl. G16H 30/40; G16H 10/60; G16H 20/10

(52) U.S. Cl. CPC G16H 30/40; G16H 10/60; G16H 20/10

(57) ABSTRACT

A method and system for predicting treatment response. Image data comprising an eye of a subject with diabetic macular edema is received. The image data may include color fundus imaging data or optical coherence tomography imaging data. A prediction model that comprises a first machine learning model generates a treatment response prediction of the subject based on the first image input. Medical data corresponding to the subject is received. A treatment response is predicted based on the image data and the medical data using a prediction model comprising a machine learning model.

### CROSS-REFERENCE TO RELATED APPLICATIONS

This application is a continuation of International Application No. PCT/US2024/059200 filed Dec. 9, 2024, which claims priority to U.S. Provisional Patent Application No. 63/642,067, filed on May 3, 2024, and entitled “Prediction of Treatment Response in Diabetic Macular Edema Patients,” and U.S. Provisional Patent Application No. 63/608,000, filed on Dec. 8, 2023, and entitled “Prediction of Treatment Response in Diabetic Macular Edema Patients,” each of which is incorporated herein by reference in its entirety.

### FIELD

The present disclosure relates generally to predicting how subjects with diabetic macular edema will respond to treatment. More particularly, the present disclosure describes methods and systems for predicting, using machine learning, treatment response with respect to a future point in time for subjects with diabetic macular edema.

### BACKGROUND

Diabetic Macular Edema (DME) is oftentimes responsible for the vision loss experienced by patients living with diabetes. With DME, excess fluid accumulates in the extracellular space within the retina in the macular area, typically in the inner nuclear layer, outer plexiform layer, Henle's fiber layer, and subretinal space. The current standard of care includes treating patients with DME using a monoclonal antibody treatment, such as faricimab, or an anti-Vascular Endothelial Growth Factor (anti-VEGF) treatment, such as ranibizumab. However, the treatment response is variable. In particular, different patients may respond differently to the treatment, with many patients not achieving desired outcomes (e.g., certain levels of vision improvement). In some cases, patients may receive more injections or injections at a higher frequency than desired. In other words, the treatment burden for these patients may be higher than desired. Accordingly, there is a need for predicting which patient has high treatment burden.

### SUMMARY

In one or more embodiments, a method is provided for predicting treatment response. Image data comprising an eye of a subject with diabetic macular edema is received. The image data may include color fundus imaging data or optical coherence tomography imaging data. A prediction model that comprises a first machine learning model generates a treatment response prediction of the subject based on the first image input.

In one or more embodiments, a method is provided for predicting treatment response. Image data comprising an eye of a subject with diabetic macular edema is received. The image data may include color fundus imaging data or optical coherence tomography imaging data. A prediction model that comprises a first machine learning model generates a treatment response prediction of the subject based on the first image input. Medical data corresponding to the subject is received. A treatment response is predicted based on the image data and the medical data using a prediction model comprising a machine learning model.

In one or more embodiments, a system comprises one or more data processors; and a non-transitory computer readable storage medium containing instructions which, when executed on the one or more data processors, cause the one or more data processors to perform one or more of the methods described herein.

In one or more embodiments, a computer-program product tangibly embodied in a non-transitory machine-readable storage medium is provided, which includes instructions configured to cause one or more data processors to perform one or more of the methods described herein.

### BRIEF DESCRIPTION OF THE DRAWINGS

The present disclosure is described in conjunction with the appended figures:

FIG. 1 is a block diagram of a treatment prediction system in accordance with one or more embodiments.

FIG. 2 is a block diagram of one embodiment of the treatment prediction system from FIG. 1 that uses optical coherence tomography (OCT) images in accordance with one or more embodiments.

FIG. 3 is a block diagram of one embodiment of the treatment prediction system from FIG. 1 that uses color fundus photography (CFP) images in accordance with one or more embodiments.

FIG. 4 is a flowchart of a process for predicting treatment response in accordance with one or more embodiments.

FIG. 5 is a segmented OCT image in accordance with one or more embodiments.

FIG. 6 is a table comparing the statistical results for machine learning models using OCT image data and segmented image data in accordance with one or more embodiments.

FIG. 7 is a block diagram of one example implementation of the treatment prediction system from FIG. 1 that includes a single modal model in accordance with one or more embodiments.

FIG. 8 is a block diagram of one example implementation of the treatment prediction system from FIG. 1 that includes a multimodal model in accordance with one or more embodiments.

FIG. 9 is a table comparing the statistical results for deep learning models using OCT image data and segmented image data in accordance with one or more embodiments.

FIG. 10 is a table including statistical results for a deep learning model using CP photographs and segmented image data in combination with clinical data in accordance with one or more embodiments.

FIG. 11 is a block diagram that illustrates a computer system in accordance with one or more embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

The embodiments described herein recognize that it may be desirable to have methods and systems for predicting a particular subject's treatment response for a diabetic macular edema (DME) treatment. The treatment may be, for example, a monoclonal antibody treatment (e.g., faricimab), an anti-Vascular Endothelial Growth Factor (anti-VEGF) treatment (e.g., ranibizumab), or another type of treatment. Anti-VEGF treatments primarily target a single pathway to reduce blood vessel leakage and proliferation. DME, however, is a multifactorial disease that can involve other angiogenic factors and inflammatory pathways not addressed with anti-VEGF monotherapy. A more recently developed treatment, faricimab, which is a bispecific monoclonal antibody, targets both VEGF (e.g., VEGF-A) and the angiopoietin-2 (Ang-2). In particular, faricimab binds both VEGF-A and Ang-2 with high affinity and specificity.

Currently, human analysts may be unable to predict how a given subject will respond to a treatment with a desired level of accuracy, speed, and/or efficiency. For example, human analysts may be unable to predict, with the desired accuracy, speed, and/or efficiency, how an individual subject's vision will improve over a selected number of months after treatment has begun.

The embodiments described herein recognize that it may be important in a healthcare setting to identify those subjects (patients) who are predicted to have at least a desired response (e.g., at least a certain level of vision improvement) to a treatment prior to administering that treatment. Such predictions may help medical professionals reduce the treatment burden imposed on a subject by a given treatment. In some cases, predicting how a subject will have responded to treatment by some future point in time can help determine whether adjustments need to be made to the dosage or injection frequency of the treatment or whether the treatment needs to be changed.

The embodiments described herein further recognize that it may be important to predict treatment responses in individuals with respect to clinical trials for purposes of enrichment, stratification, and/or covariate adjustment in primary analysis. For example, it may be important to identify and select participants who are most likely to benefit from or respond to a treatment offered in a clinical trial, which may include participants or a sub-population that does not respond well or is predicted to not respond well to the current available treatment options; classify or segment individuals into groups or “strata” based on their predicted response to a treatment; and/or incorporate the predicted response to a treatment (reference arm treatment) as the covariate. Covariate adjustment is a statistical process that uses baseline measurements from participants to estimate treatment effects in clinical trials.

Thus, the embodiments described herein provide methods and systems for predicting the response of a subject to treatment at a future point in time. The methods and systems described herein use machine learning (e.g., one or more machine learning models) to process various types of input data to generate a predicted treatment response.

The input data may include medical data including one or more of treatment information (e.g., treatment regimen data and a treatment naïve indication or label), clinical data, demographic data, and diabetic status information. The input data may also include image data of a subject's eye. For example, the image data may include an OCT image and/or or a color fundus photography (CFP) image of a subject's retina.

In some embodiments, the predicted treatment response may be based on the input data. In some embodiments, the input data, including image data (e.g., an OCT image or a CFP image) and/or medical data, may be input into an image preprocessor model to generate segmented image data (e.g., a segmented OCT image or a segmented CFP image). In some embodiments, the image preprocessor model may include a machine learning model that identifies one or more layers or features of the retina shown in the image data. The segmented image data, such as for example a measurement associated with a retinal element, may then be input into a prediction model to generate a predicted treatment response. In some embodiments, the prediction model may include a machine learning model that predicts a future measurement of the eye that corresponds to or may be used to determine the subject's response to the treatment. In some embodiments, a segmented OCT image or segmented CFP image may not be input into the prediction model and instead the original OCT image or original CFP image, segmented image data, and/or medical data may be directly input into the prediction model. In some embodiments, the prediction model receives image input from multi-visits such that the image input includes a first image input from a first period of time and a second image input from a second period of time for one subject. Accordingly, and in some embodiments, input for the prediction model may include images, such as for example, an image; segmented images, such as for example a segmented OCT image; retinal image maps, such as for example a thickness map or intensity map based on the segmented image; and/or image data such as for example measurements, quantities, and/or values of retinal elements identified in a segmented image or derived from a segmented image. In some embodiments, a map is a measurement.

In some embodiments, the predicted treatment response may be a predicted visual acuity (e.g., best corrected visual acuity (BCVA)) measurement, a predicted macular thickness, or some other predicted vision health metric for a future point in time. The future point in time may be, for example, without limitation, 4 weeks, 6 weeks, 18 weeks, 24 weeks, 1 month, 3 months, 4 months, 5 months, 6 months, 1 year, 2 years, or some other number of days, weeks, months, or years after a reference point in time. The reference point in time may be, for example, a baseline point in time with respect to treatment or some point in time after a first dose of treatment. In some embodiments, the predicted treatment response comprises multiple prediction outputs for different points in time.

Some of the embodiments described herein may be directed to methods and systems for preparing the data that will be used for predicting the treatment outcome of the subject. In some embodiments, predicting the treatment outcome for the subject may be based on image data and/or medical data of the subject. For example, the image data may include an OCT image or CFP image and the medical data may include a baseline visual acuity (e.g. best corrected visual acuity (BCVA)) measurement, a baseline central subfield thickness (CST), a baseline diabetic retinopathy severity scale (DRSS) score, sex, age, diabetes type, and/or a treatment naïve label. In some cases, instead of simply inputting the image data and medical data into the image preprocessor model, the image data and medical data may be combined or matched in a meaningful way, thereby improving the ability of the image processor model to segment the image or the ability of the prediction model to predict the treatment outcome. For example, the medical data of the subject may be matched with the image data at a subject level or at an eye level. Thus, combining or matching the image data and the medical data may result in a more useful input to the image preprocessor model to generate a more accurate segmentation and/or to the prediction model to generate a more accurate prediction.

Predicting subject-specific treatment response for a given treatment may help improve overall treatment management of DME. For example, more accurately predicting a specific subject's treatment response may help in the development of more tailored or customized treatment regimens for individual subjects. By predicting whether the treatment protocol will result in a desired response, the healthcare provider may be able to recommend a treatment plan that will result in the best outcome for the subject while minimizing the use of ineffective treatments.

In another example, predicting how the specific subject will respond to a particular treatment may help determine the subjects that should be included in a clinical trial. If the subject is likely to not respond to treatment(s) that are currently available, it may be advantageous to include that subject in a clinical trial for a new treatment. Thus, improving the treatment response prediction for specific subjects to currently available treatments may improve the creation of clinical trial populations in testing new treatment(s).

In some embodiments, the embodiments described herein provide one or more technical benefits, which may include, for example, without limitation, improving the performance of a model (e.g., ability of the prediction model to predict the treatment outcome).

### II. Overview of Example Treatment Response Prediction System for DME

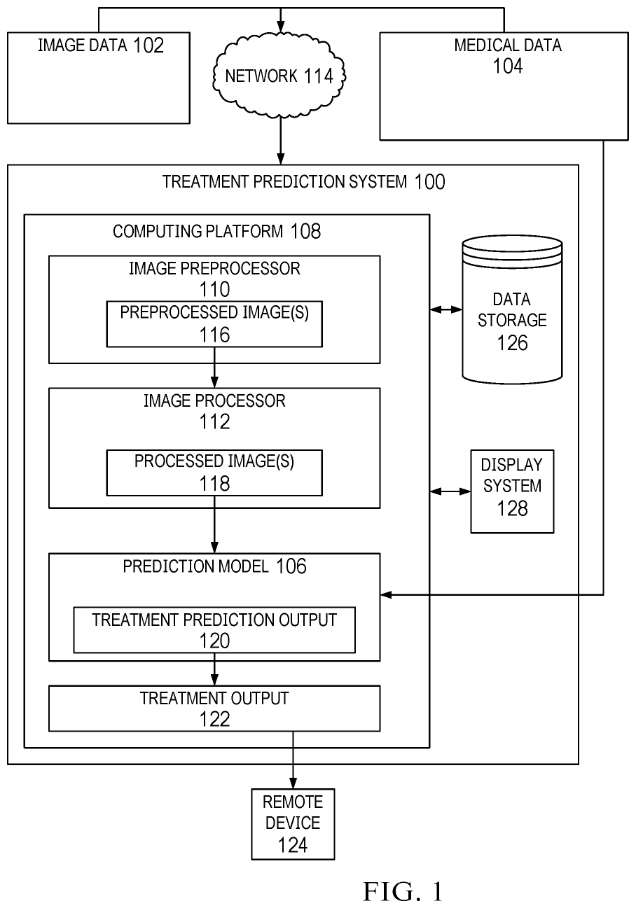

Referring now to the figures, FIG. 1 is a block diagram of a treatment prediction system 100 in accordance with various embodiments. The treatment prediction system 100 may be used to predict a treatment response for a subject with diabetic macular edema (DME). In some embodiments, the treatment prediction system 100 is single modal or unimodal based on image data 102 and in other embodiments the treatment prediction system is multimodal based on the image data 102 and medical data 104. In particular, the treatment prediction system 100 may include a prediction model 106 that predicts how the eye of a subject with DME will respond to treatment, as determined at a future point in time. For example, the treatment prediction system 100 may be used to predict how the eye will respond to treatment after a selected number of days, weeks, months, or years (e.g., 4 weeks, 6 weeks, 18 weeks, 24 weeks, 3 months, 6 months, 9 months, 1 year, 2 years, etc.).

In some embodiments, the image data 102 undergoes a number of processing steps before it is sent to the prediction model 106. As illustrated in FIG. 1, the treatment prediction system 100 includes a computing platform 108 configured to store and execute an image preprocessor 110, an image processor 112, and the prediction model 106. While the image preprocessor 110, the image processor 112, and the prediction model 106 are illustrated as being stored and executed using the same computing platform (i.e., the computing platform 108), in some embodiments, one or more of the image preprocessor 110, the image processor 112, and the prediction model 106 are stored and executed using a computing platform that is different from the computing platform 108.

Generally, the image preprocessor 110 receives or accesses, for example using a network 114, the image data 102 and performs a set of preprocessing operations on the image data 102 to form preprocessed image(s) 116. The image data 102 may be sent as input into the image preprocessor 110, retrieved by the image preprocessor 110 from storage, or accessed in some other manner. In some embodiments, some or all of the medical data 104 may be input into the image preprocessor 110 with the image data 102. In some embodiments, the medical data 104 and the image data 102 may be combined or matched before being input into the image preprocessor 110. In other embodiments, the image preprocessor 110 matches the medical data 104 and the image data 102, for example based on metadata associated with each of the medical data 104 and the image data 102. The set of processing operations may include, for example, without limitation, at least one of a normalization operation, a scaling operation, a resizing operation, a horizontal flipping operation, a vertical flipping operation, a cropping operation, a rotation operation, a noise filtering operation, or some other type of preprocessing operation. The image preprocessor 110 may be implemented using hardware, software, firmware, or a combination thereof. In one or more embodiments, the image preprocessor 110 may be implemented within the computing platform 108 but in other embodiments at least a portion of (e.g., a module of) the image preprocessor 110 is implemented within an imaging system that generates the image data 102.

In some embodiments, the preprocessed image(s) 116 require additional processing before being sent to the prediction model 106. As such, the preprocessed image(s) 116 may be sent to the image processor 112, which generates processed image(s) 118 using the preprocessed image(s) 116. The preprocessed image(s) 116 may be sent as input into the image processor 112, retrieved by the image processor 112 from storage, or accessed in some other manner. In some embodiments, the image processor 112 identifies retinal features on the processed image(s) and may generate retinal data based on the identified retinal features. The identified retinal features and related retinal data may be included in the processed image(s) 118 and may be used by the prediction model 106. The image processor 112 may be implemented using hardware, software, firmware, or a combination thereof.

In some embodiments, the prediction model 106 uses the processed image(s) 118 from the image processor 112 to generate a treatment prediction output 120, which predicts the treatment response for a subject with DME. In some embodiments, at least some of the medical data 104 and/or the image data 102 may be directly input into the prediction model 106. For example, in some embodiments, the medical data 104 and/or the image data 102 is input into the prediction model 106. In one or more embodiments, the treatment prediction output 120 may be defined using one or more different types of vision-related outcomes, such as for example vision acuity, a predicted macular thickness, one or more other types of predicted visual outcome metrics, or a combination thereof. In some examples, the treatment prediction output 120 may be a predicted measurement at a future point in time, such as for example a predicted best corrected visual acuity (BCVA) at the future point in time (e.g., BCVA predicted at week 4, week 24, or 1 year post-treatment); a predicted central subfield thickness (CST) at the future point in time (e.g., CST predicted at week 24 post-treatment); and/or a predicted difference between the CST at a first point in time, such as at baseline, and a second point in time, such as a future point in time. The future point in time may be, for example, 4 weeks, 6 weeks, 18 weeks, 24 weeks, 1 month, 3 months, 4 months, 5 months, 6 months, 1 year, 2 years or some other amount of time after treatment has begun. In some embodiments, the prediction model 106 may include a RETFound model, a ResNet model, a linear regression or elastic net model, a random forest model, a support vector machine model, a regression model, a Extreme Gradient Boosting (XGBoost) model, another type of machine learning model, a convolutional neural network (CNN) such as for example a ResNet50 model pretrained on ImageNet, a single modal OCT foundation model such as for example RETFound, a multi-modal OCT foundation model such as for example RETFound, a vision transformer based deep learning model, or other type of deep learning model. In some embodiments, the prediction model 106 uses image input from multi-visits such that the image data includes a first image data from a first period of time and a second image data from a second period of time for one subject.

In some embodiments, the treatment prediction output 120 includes an identification of the subject associated with the image data 102 as a subject with a predicted measurement that exceeds or does not exceed a threshold predicted measurement. In some embodiments, the threshold predicted measurement may be a minimum change in BCVA at a future point in time. In some embodiments, the treatment prediction output 120 is a categorial outcome prediction or a continuous outcome prediction. In some embodiments, the treatment prediction output 120 is used to generate a treatment output 122.

In some embodiments, the treatment output 122 may include a treatment recommendation for the subject based on the treatment prediction output 120. In some embodiments, and when the subject is identified as having a predicted measurement that exceeds the threshold predicted measurement and with respect to a first treatment regimen, then the treatment output 122 may include a recommendation to administer the first treatment regimen. That is, when exceeding the threshold predicted measurement is associated with a desired response to the first treatment regimen and the subject exceeds the threshold predicted measurement, then the treatment output 122 includes a recommendation to administer the first treatment regimen. However, in other embodiments and when the subject is identified as having a predicted measurement that does not exceed a threshold predicted measurement, then the treatment output 122 may include a recommendation that is different from administering the first treatment regimen. That is, when the subject is predicted by the prediction model 106 to have a less desirable response to the first treatment regimen, then the treatment output 122 may include administering a treatment regime that is different from the first treatment regimen.

In some embodiments, when the subject is identified as having a predicted measurement—to a monoclonal antibody treatment, such as Faricimab—that exceeds the threshold predicted measurement, then the treatment output 122 may include a recommendation to administer the monoclonal antibody treatment, such as Faricimab. In some embodiments, when the subject is identified as having a predicted measurement—to an anti-Vascular Endothelial Growth Factor (anti-VEGF) treatment, such as ranibizumab—that exceeds the threshold predicted measurement, then the treatment output 122 may include a recommendation to administer the anti-Vascular Endothelial Growth Factor (anti-VEGF) treatment, such as ranibizumab. In some embodiments, the treatment output 122 may include, for example, the treatment prediction output 120.

In some embodiments, the prediction model 106 uses image input from multi-visits such that the image data includes a first image data from a first period of time and a second image data from a second period of time for one subject. When the second period of time is after the first treatment regimen has begun and when the subject is identified as having a predicted measurement that exceeds the threshold predicted measurement, then the treatment output 122 includes a recommendation to continue administering the first treatment regimen. However, if the subject is identified as having a predicted measurement that does not exceed the threshold predicted measurement, then the treatment output 122 may include a recommendation to change the treatment regimen from the first treatment regimen.

In some embodiments, the treatment output 122 includes other types of information. For example, in some cases, the treatment output 122 includes a clinical trial recommendation, the treatment recommendation, or both. A clinical trial recommendation may be a recommendation to include or exclude the subject from a clinical trial. The treatment recommendation may be a recommendation to change the type of treatment that will be given to the subject, adjust the treatment regimen (e.g., injection frequency, dosage, etc.) for the treatment, consider a new type of treatment, add a new treatment to the existing treatment regimen, or alter the treatment plan for the subject in some other manner.

In some embodiments, the treatment output 122 is sent to a remote device 124 via the network 114. The treatment prediction system 100 also includes a data storage 126 and a display system 128. The data storage 126 and the display system 128 are each in communication with the computing platform 108. In some examples, the data storage 126, the display system 128, or both may be considered part of or otherwise integrated with the computing platform 108. Thus, in some examples, the computing platform 108, the data storage 126, and the display system 128 may be separate components in communication with each other, but in other examples, some combination of these components may be integrated together. In one or more embodiments, the computing platform 108 includes a single computer (or computer system) or multiple computers in communication with each other. In other examples, the computing platform 108 takes the form of a cloud computing platform, a mobile computing platform (e.g., a smartphone, a tablet, etc.), or a combination thereof.

At least a portion of treatment output 122 or a graphical representation of at least a portion of treatment output 122 may be displayed on the display system 128. In some embodiments, at least a portion of treatment output 122 or a graphical representation of at least a portion of treatment output 122 is sent to remote device 124 (e.g., a mobile device, a laptop, a server, a cloud, etc.).

Predicting subject-specific treatment response using the treatment prediction system 100 for a given treatment is more accurate and may help improve overall treatment management of DME. For example, more accurately predicting a specific subject's treatment response using the treatment prediction system 100 may help in the development of more tailored or customized treatment regimens for individual subjects. By predicting, using the treatment prediction system 100, whether the treatment protocol will result in a desired response, then a healthcare provider may be able to recommend a treatment plan that will result in the best outcome for the subject while minimizing the use of ineffective treatments.

In another example, predicting how the specific subject will respond to a particular treatment using the treatment prediction system 100 may help determine the subjects that should be included in a clinical trial. If the subject is likely to respond to the treatment being tested by the clinical trial, it may be advantageous to include them in the clinical trial. On the other hand, if a subject is unlikely to respond to the treatment being tested by the clinical trial, it may not be advantageous to include them in the clinical trial. Thus, improving the treatment response prediction for specific subjects may improve the creation of clinical trial populations. Further, the treatment output 122 may be used in clinical trials for enrichment, stratification, or covariate adjustment.

Additionally, using the image preprocessor 110 and/or the image processor 112 to process the input data may reduce the overall computing resources that would be otherwise needed to make such predictions and/or general determinations/recommendations about clinical trials, treatment management, or both. By generating more effective input data using the image preprocessor 110 and/or the image processor 112, for example by combining or matching image data with the medical data, the image preprocessor 110, the image processor 112 and/or the prediction model 106 may be able to more efficiently generate a more accurate output, saving computing resources.

In some embodiments, the treatment prediction system 100 predicts treatment response for patients with better accuracy and consistency than expert human graders. The treatment prediction system 100 provides a technical effect of improving accuracy, reducing the overall computing resources, and/or reducing the time needed to predict a specific subject's predicted treatment response. Further, using the treatment prediction system 100 may help improve overall treatment management of DME as compared to other methods and systems.

In some embodiments, the treatment prediction system 100 provides a technical improvement to the field of DME treatment and/or the technical field of predicting a specific subject's predicted treatment response. As noted above, the treatment prediction system 100 predicts treatment response for patients with better accuracy and consistency than expert human graders and may reduce the overall computing resources and/or time needed to predict treatment response for subjects.

### II.A. Example System for Predicting Treatment Response for DME Using OCT Images

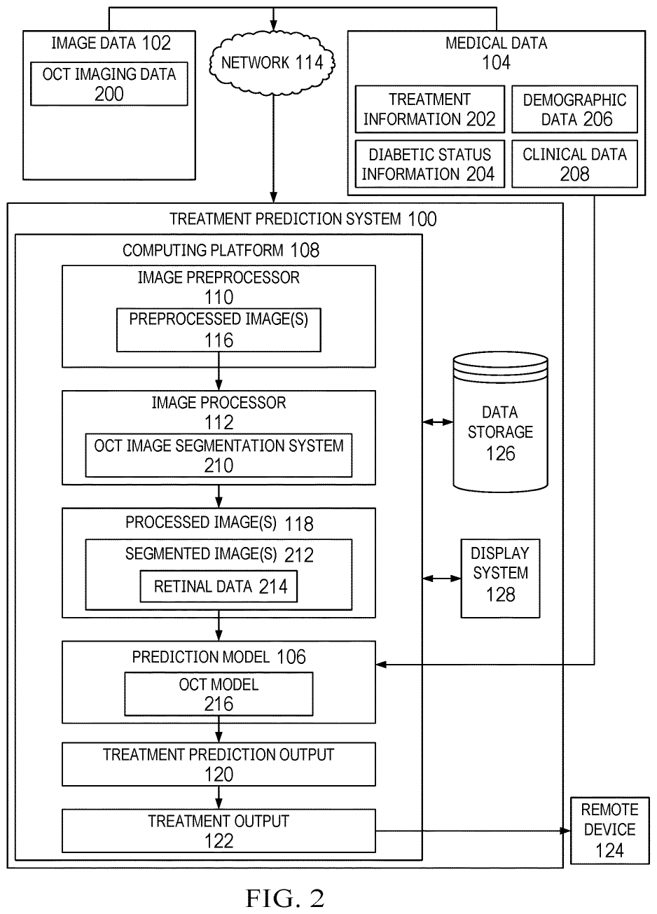

FIG. 2 is a block diagram of an embodiment of the treatment prediction system 100 that uses OCT imaging data 200 as the image data 102. In some embodiments, the treatment prediction system 100 is single modal (e.g., based on the OCT image data 200) and in other embodiments the treatment prediction system is multimodal (e.g., based on the OCT image data 200 and the medical data 104). In particular, the treatment prediction system 100 may include a prediction model 106 that predicts, using the OCT image data 200, how the eye of a subject with DME will respond to treatment, as determined at a future point in time.

As illustrated, the image data 102 is or includes include the OCT imaging data 200. The OCT imaging data 200 may include one or more raw images obtained directly via an imaging system that generated the raw images or the OCT imaging data 200 may include preprocessed raw images. The OCT imaging data 200 may include, for example, without limitation, time domain OCT images (TD-OCT), spectral domain OCT (SD-OCT) images, two-dimensional OCT images (e.g., OCT B scans), three-dimensional OCT images (e.g., OCT volume images), OCT angiography (OCT-A) images, or a combination thereof. An OCT volume may itself be comprised of multiple OCT B-scans. OCT B-scans may include, for example, without limitation, 10s, 100s, 1000s, 10,000s, or some other number of OCT B-scans. An OCT B-scan may also be referred to as an OCT slice image or a cross-sectional OCT image. The OCT imaging data 200 may be generated using an OCT imaging system or OCT scanner. The OCT imaging system can be a large tabletop configuration used in clinical settings, a portable or handheld dedicated system, or a “smart” OCT system incorporated into user personal devices such as smartphones. In some cases, the OCT imaging system may include an image denoiser that is configured to remove noise and other artifacts from a raw OCT volume image to generate an OCT volume.

In some embodiments, the medical data 104 may include treatment information 202. The treatment information 202 may include treatment regimen data. Generally, treatment regimen data identifies the treatment for which the response is being predicted. Treatment regimen data may identify a treatment (e.g., a monoclonal antibody treatment, such as faricimab, or an anti-Vascular Endothelial Growth Factor (anti-VEGF) treatment, such as ranibizumab) for treating the subject and at least one of an administration frequency, a dosage schedule, a monitoring schedule, or a combination thereof for the treatment. The administration frequency may be, for example, an injection frequency (e.g., for intravitreal injections). The dosage schedule may be, for example, the dosage amount per injection, which may remain constant or may change over time (e.g., a different dosage for earlier injections as compared to later injections). The monitoring schedule may be, for example, a schedule for one or more monitoring visits or imaging sessions to assess treatment progress. In this manner, treatment regimen data identifies the treatment and may additionally provide information about the burden associated with such treatment. In some cases, the treatment information 202 may also include treatment naïve indication. Generally, the treatment naïve indication provides an indication of whether the subject has been previously treated for DME or not (i.e., is treatment naïve).

In some embodiments, the medical data 104 may include diabetic status information 204. The diabetic status information 204 may include, for example, without limitation, at least one of a diabetes type, a duration of diabetes, a duration of DME, a diabetic retinopathy severity score (e.g., DRSS score as developed the Early Treatment Diabetic Retinopathy Study (ETDRS)).

In some embodiments, the medical data 104 may include demographic data 206. The demographic data 206 may include, for example, age, sex, one or more other types of demographic variables, or a combination thereof.

In some embodiments, the medical data 104 may include clinical data 208. The clinical data 208 may include a visual acuity measurement, a blood sugar level, one or more blood pressure levels, a body mass index, an intraocular pressure, or another type of clinical parameter or measurement. The visual acuity measurement may be, for example, a BCVA at baseline (with respect to treatment). The blood sugar level may be, for example, the blood sugar level as determined by a hemoglobin A1C test. The one or more blood pressure levels may include a systolic blood pressure (SBP), a diastolic (DBP), or both. The clinical data 208 may include data corresponding to a reference point in time. The reference point in time may, in some cases, be referred to as a baseline point in time that is the baseline after which treatment response is being predicted. This baseline point in time may be, for example, a baseline point in time with respect to treatment. The baseline (with respect to treatment) point in time may be, for example, a point in time prior to treatment, the same day as a treatment dose (e.g., a first treatment dose), or some other type of baseline with respect to treatment. In some cases, the reference point in time is a point in time after a first dose of treatment such as, for example, 4 weeks, 6 weeks, 18 weeks, 24 weeks, 3 months, 6 months, 9 months, 1 year, 2 years, or some other amount of time after the first dose of treatment. Generally, the clinical data 208 corresponding to the reference point in time may include data generated at the reference point in time, data generated within a selected range (e.g., within a selected number of days, weeks, or months) of the reference point in time, or both.

In this example, the image preprocessor 110 is configured or programmed to receive and perform a set of preprocessing operations on the OCT imaging data 200 to form the preprocessed image(s) 116. In some embodiments, some or all of the medical data 104 may be input into the image preprocessor 110 with the OCT imaging data 200. In some embodiments, the medical data 104 and OCT imaging data 200 may be combined or matched before being input into the image preprocessor 110. The set of processing operations may include, for example, without limitation, at least one of a normalization operation, a scaling operation, a resizing operation, a horizontal flipping operation, a vertical flipping operation, a cropping operation, a rotation operation, a noise filtering operation, or some other type of preprocessing operation. In one or more embodiments, the image preprocessor 110 may be implemented within the computing platform 108 but in other embodiments at least a portion of (e.g., a module of) the image preprocessor 110 is implemented within an imaging system, which may be or include an OCT imaging system. In some embodiments, the preprocessed image(s) 116 are sent to the image processor 112.

As illustrated in FIG. 2 and when the treatment prediction system 100 uses the OCT imaging data 200, the image processor 112 may be or include an OCT image segmentation system 210. In some embodiments, the OCT image segmentation system 210 receives an OCT volume, from the image preprocessor 110 or elsewhere, and extracts B-scans from the OCT volume according to a distance from a center B-scan. For example, the OCT image segmentation system 210 may extract all B-scans within 0.5 mm of the middle B-scan. In some embodiments, the OCT image segmentation system 210 is a segmentation system that generates segmented image(s) 212 using the preprocessed image(s) 116, but the segmentation system may also generate the segmented image(s) 212 using the OCT imaging data 200. For example, the OCT imaging data 200 may include the OCT volume, which is an input to the OCT image segmentation system 210.

The segmented image(s) 212 may be used to identify various retinal elements within an OCT B-scan. One or more of the segmented image(s) 212 may be generated from the OCT imaging data 200 according to one or more techniques as described in International Publication No. WO2023205511A1, which is incorporated by reference herein in its entirety. Moreover and in some embodiments, the OCT image segmentation system 210 is or includes one or more of the systems for automated retinal segmentation as described in International Publication No. WO2023205511A1.

A retinal element may be comprised of at least one of a retinal layer element or a retinal pathological element. Detection and identification of one or more retinal layer elements may be referred to as layer element (or retinal layer element) segmentation. Detection and identification of one or more retinal pathological elements may be referred to as pathological element (or retinal pathological element) segmentation. The OCT image segmentation system 210 identifies one or more retinal (e.g., retina-associated) elements on the segmented image(s) 212 using one or more graphical indicators. For example, one or more color indicators, shape indicators, pattern indicators, shading indicators, lines, curves, markers, labels, tags, text features, other types of graphical indicators, or a combination thereof may be used to identify the portion(s) (e.g., by pixel) of an OCT image that have been identified as a retinal element. As one specific example, a group of pixels may be identified as capturing a particular retinal fluid (e.g., IRF or SRF). A segmented image may identify this group of pixels using a color indicator. For example, each pixel of the group of pixels may be assigned a color that is unique to the particular retinal fluid and thereby assigns each pixel to the particular retinal fluid. As another example, the segmented image(s) 212 may identify the group of pixels by applying a patterned region or shape (continuous or discontinuous) over the group of pixels.

A retinal layer element may be, for example, a retinal layer or a boundary associated with a retinal layer. Examples of retinal layers include, but are not limited to, the internal limiting membrane (ILM) layer, the retinal nerve fiber layer, the ganglion cell layer, the inner plexiform layer, the inner nuclear layer, the outer plexiform layer (OPL), the outer nuclear layer, the external limiting membrane (ELM) layer, the photoreceptor layer(s), the retinal pigment epithelial (RPE) layer, a RPE detachment, the Bruch's membrane (BM) layer, the choriocapillaris layer, the choroidal stroma layer, the ellipsoid zone (EZ), and other types of retinal layers. In some cases, a retinal layer may be comprised of one or more layers. As one example, a retinal layer may be the interface between an outer plexiform layer and Henle's fiber layer (OPL-HFL). A boundary associated with a retinal layer may be, for example, an inner boundary of the retinal layer, an outer boundary of the retinal layer, a boundary associated with a pathological feature of the retinal layer (e.g., an inner or outer boundary of detachment of the retinal layer), or some other type of boundary. For example, a boundary may be an inner boundary of a RPE (IB-RPE) detachment layer, an outer boundary of the RPE (OB-RPE) detachment layer, or another type of boundary.

A retinal pathological element may include, for example, fluid (e.g., a fluid pocket), cells, solid material, or a combination thereof that evidences a retinal pathology (e.g., disease or condition such as diabetic macular edema or age-related macular degeneration (AMD)). For example, the presence of certain retinal fluids may be a sign of neovascular AMD. Examples of retinal pathological elements include, but are not limited to, intraretinal fluid (IRF), subretinal fluid (SRF), fluid associated with pigment epithelial detachment (PED), hyperreflective material (HRM), subretinal hyperreflective material (SHRM), intraretinal hyperreflective material (IHRM), hyperreflective foci (HRF), a retinal fluid pocket, drusen, and fibrosis. In some cases, a retinal pathological element may be a disruption (e.g., discontinuity, delamination, loss, etc.) of a retinal layer or retinal zone. For example, the disruption may be of the ellipsoid zone, of the ELM, of the RPE, or of another layer or zone. The disruption may represent damage to or loss of cells (e.g., photoreceptors) in the area of the disruption. In some examples, a retinal pathological element may be clear IRF, turbid IRF, clear SRF, turbid SRF, some other type of clear retinal fluid, some other type of turbid retinal fluid, or a combination thereof. The OCT image segmentation system 210 may include one or more machine learning models (e.g., one or more deep learning models, such as, but not limited to CNNs) that can perform retinal segmentation on the image data 102 and/or the processed image(s) 116 to generate segmented image data. The OCT image segmentation system 210 may further include one or more machine learning models (e.g., one or more deep learning models, such as, but not limited to CNNs) that can extract retinal data 214 from the segmented image(s) 212 (segmented image data).

Additionally, a retinal pathological element may include a characteristic or subtype of one of the fluids (e.g., IRF, SRF, fluid associated with PED), materials (e.g., HRM, SHRM, IHRM), lesions (e.g., HRF, SHRM lesions), or disruptions. In particular, examples of retinal pathological elements may include characteristics and/or subtypes of the different types of elements and disruptions described above that can be detected and identified via retinal segmentation. For example, whether a retinal fluid is clear or turbid may be detectable and identifiable characteristic of the retinal fluid. Accordingly, in some examples, a retinal pathological element may be clear IRF, turbid IRF, clear SRF, turbid SRF, some other type of clear retinal fluid, some other type of turbid retinal fluid, or a combination thereof. In some cases, for SHRM, shape characteristics (e.g., tall SHRM, dome-shaped SHRM at the foveal center, flat SHRM near the foveal center, dysmorphic, etc.), boundary characteristics (e.g., ill-defined SHRM, well-defined SHRM), reflectivity (e.g., increased reflectivity or other levels of reflectivity), layering characteristics (e.g., hyperreflective bands in SHRM lesions), and lesion characteristics (e.g., the height, width, and/or area of SHRM lesions) may be examples of retinal pathological elements that may be detected and identified via retinal segmentation.

Additionally, the OCT image segmentation system 210 may identify the Bruch's membrane (BM) layer in each of the B-scans and flatten each of the B-scans along the Bruch's membrane. In some examples and when the prediction model 106 requires RGB based images, one of the image preprocessor 110 and/or the OCT image segmentation system 210 may generate repeating B-scans along channel dimensions or adjoining neighboring B-scans along the R and B channels. The segmented image(s) 116 may be a representation of a raw or preprocessed image (e.g., OCT B scan, OCT volume image) that identifies the one or more retinal elements or may be the raw or preprocessed image on which the one or more retinal elements have been identified.

The segmented image(s) 212 may include the retinal data 214. In some embodiments, the OCT image segmentation system 210 generates the retinal data 214. The retinal data 214 may include values for various retinal features relating to one or more pathological elements of the retina, one or more layers of the retina, or both. The retinal data 214 may include, for example, without limitation, feature data extracted from the segmented image data described above. For example, feature data may be extracted for one or more retinal elements identified in the segmented image data. This feature data may include values for any number of or combination of features (e.g., quantitative features). These features may include pathology-related features, layer-related volume features, layer-related thickness features, or a combination thereof. Examples of features include, but are not limited to, a maximum retinal layer thickness, a minimum retinal layer thickness, an average retinal layer thickness, a maximum height of a boundary associated with a retinal layer, a volume of a retinal fluid pocket, a length of a fluid pocket, a width of a fluid pocket, a number of retinal fluid pockets, and a number of hyperreflective foci. Thus, at least some of the features may be volumetric features. For example, the retinal data 214 may be derived for each selected OCT image (e.g., single OCT B scan) and then combined to form volume-wide values. In some embodiments, the retinal data 214 may be derived for each selected OCT image and then aggregated over the ETDRS grid. In some embodiments, the retinal data 214 includes a mean retinal thickness between ILM and BM, an IRF volume, an SRF volume in the center 1 mm and inner subfields.

As illustrated in FIG. 2 and when the treatment prediction system 100 uses the OCT imaging data 200, the prediction model 106 may be or include an OCT model 216 to generate the treatment prediction output 120 based on the segmented image(s) 212 alone or in combination with the medical data 104. In some embodiments, at least some of the input data, which includes the OCT imaging data 200 and the medical data 104, may be directly input into the OCT model 216. For example, in some embodiments, the medical data 104 and/or the OCT imaging data 200 is input into the OCT model 216. In some embodiments, the OCT model 216 may include a RETFound model, a ResNet model, a elastic net model, a random forest model, a support vector machine model, a regression model, a Extreme Gradient Boosting (XGBoost) model, another type of machine learning model, a convolutional neural network (CNN) such as for example a ResNet50 model pretrained on ImageNet, a single modal OCT foundation model such as for example RETFound, and a multi-modal OCT foundational model such as for example RETFound.

The OCT model 216 uses the segmented image(s) 212 to generate the treatment prediction output 120, which predicts treatment response for a subject with DME. For example, the OCT model 216 may provide a predicted 1-year BCVA for the subject associated with the segmented image(s) 212 or provide a prediction for some other future point in time. In other examples, the treatment prediction output 120 generated by the OCT model 216 includes a predicted difference between a central subfield thickness (CST) at baseline and a future point in time. The future point in time may be, for example, 4 weeks, 6 weeks, 18 weeks, 24 weeks, 1 month, 3 months, 4 months, 5 months, 6 months, 1 year, 2 years or some other amount of time after treatment has begun.

In some embodiments, the OCT model 216 generates the treatment prediction output 120 described in FIG. 1 based on the OCT imaging data 200. In some embodiments, the OCT model 216 generates the treatment prediction output 120 described in FIG. 1 based on OCT imaging data 200 from a baseline timepoint and one or more timepoints after the baseline timepoint. That is, the OCT imaging data 200 may be associated with a subject at a first point of time and a second point of time that is later than the first point of time.

While FIG. 2 illustrates an image preprocessor 110, the image processor 112, and the OCT model 216 as separate components, in some embodiments the OCT model 216 is an end-to-end deep learning model configured to use raw OCT imaging data 200 and produce the treatment prediction output 120 and/or the treatment output 122.

In some embodiments, the treatment prediction output 120 generated by the OCT model 216 is used to generate the treatment output 122 described in FIG. 1.

In some embodiments, the treatment prediction system 100 that includes the OCT model 216 predicts treatment response for patients with better accuracy and consistency than expert human graders. The treatment prediction system 100 that includes the OCT model 216 provides a technical effect of improving accuracy, reducing the overall computing resources, and/or reducing the time needed to predict a specific subject's predicted treatment response. Further, using the treatment prediction system 100 that includes the OCT model 216 may help improve overall treatment management of DME as compared to other methods and systems.

In some embodiments, the treatment prediction system 100 that includes the OCT model 216 provides a technical improvement to the field of DME treatment and/or the technical field of predicting a specific subject's predicted treatment response. As noted above, the treatment prediction system 100 that includes the OCT model 216 predicts treatment response for patients with better accuracy and consistency than expert human graders and may reduce the overall computing resources and/or time needed to predict treatment response for subjects.

### II.B. Example System for Predicting Treatment Response for DME Using CFP Images

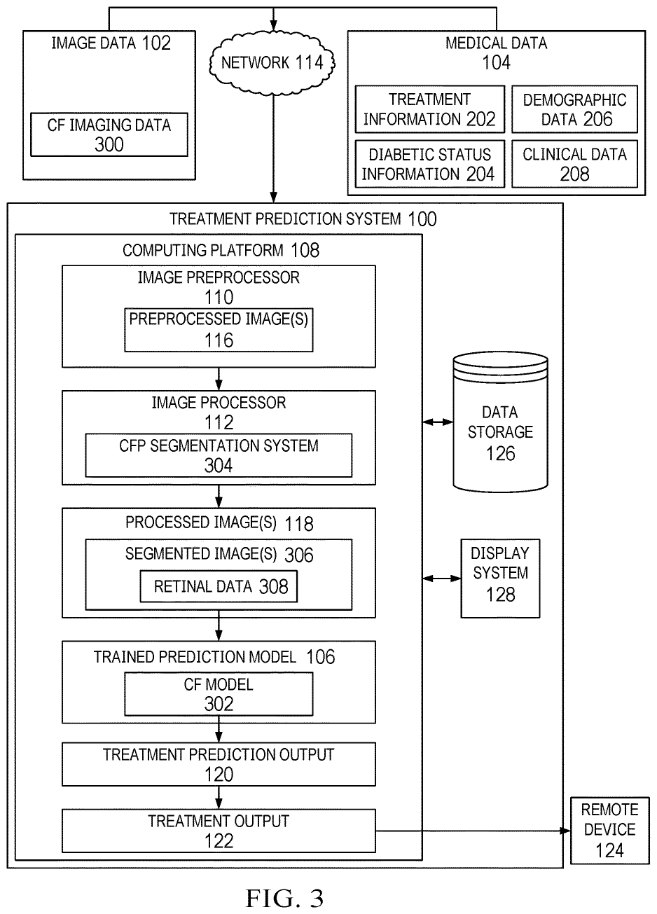

FIG. 3 is a block diagram of an embodiment of the treatment prediction system 100 that uses CF imaging data 300 as the image data 102. In some embodiments, the treatment prediction system 100 is single modal (e.g., based on the CF imaging data 300) and in other embodiments the treatment prediction system 100 is multimodal (e.g., based on the CF imaging data 300 and the medical data 104). In particular, the treatment prediction system 100 may include a CF model 302 that predicts, using the CF imaging data 300, how the eye of a subject with DME will respond to treatment, as determined at a future point in time.

As illustrated, the image data 102 is or includes include the CF imaging data 300. In some embodiments, the CF imaging data 300 may include, for example, one or a plurality of fields of view (or fields) of color fundus images generated using a color fundus imaging technique (also referred to as color fundus photography). In one or more embodiments, the CF imaging data 300 includes seven-field color fundus imaging data and/or four-wide color fundus imaging data. In some embodiments, each field of view comprises a color fundus image.

In some embodiments, the medical data 104 may include the treatment information 202, the diabetic status information 204, the demographic data 206, and/or the clinical data 208 as described in FIG. 2.

In this example, the image preprocessor 110 is configured or programmed to receive and perform a set of preprocessing operations on the CF imaging data 300 to form the preprocessed image(s) 116. In some embodiments, some or all of the medical data 104 may be input into the image preprocessor 110 with the CF imaging data 300. In some embodiments, the medical data 104 and the CF imaging data 300 may be combined or matched before being input into the image preprocessor 110. The set of processing operations may include, for example, without limitation, an image standardization procedure to generate a set of standardized image data. In some embodiments, the at least one image standardization procedure comprises one or more of: a field detection procedure, a central cropping procedure, a foreground extraction procedure, a region extraction procedure, a central region extraction procedure, an adaptive histogram equalization (AHE) procedure, and a contrast limited AHE (CLAHE) procedure. In some embodiments, the image preprocessor 110 is configured to perform any at least 1, 2, 3, 4, 5, 6, or 7, or at most any 7, 6, 5, 4, 3, 2, or 1 of the aforementioned procedures. In one or more embodiments, the image preprocessor 110 may be implemented within the computing platform 108 but in other embodiments at least a portion of (e.g., a module of) the image preprocessor 110 is implemented within an imaging system, which may be or include an CF imaging system. In some embodiments, the preprocessed image(s) 116 are sent to the image processor 112.

In one example, the image preprocessor 110 and/or the image processor 112 comprises an auto-field-classifier that selects field 2 images centered on macula and the image preprocessor 110 and/or the image processor 112 calculates, from the selected field 2 images, the width and height of the minimum-sized image, and crops all images to that size. In some embodiments, the image preprocessor 110 and/or the image processor 112 creates a montage of CFP images for model prediction.

As illustrated in FIG. 3 and when the treatment prediction system 100 uses the CF imaging data 300, the image processor 112 may be or include a CFP segmentation system 304. In some embodiments, the CFP segmentation system 304 is a segmentation system that generates segmented image(s) 306 using the preprocessed image(s) 116, but the CFP segmentation system 304 may also generate the segmented image(s) 306 using the CF imaging data 300.

The segmented image(s) 306 may be used to identify various retinal elements within a CFP image. For example, the segmented image(s) 306 may identify a pathological element of the retina such as for example a DR lesion feature that may include for example a cotton wool spot, a hard exudate, a hemorrhage, and a microaneurysm. The CFP segmentation system 304 may identify one or more retinal (e.g., retina-associated) elements on the segmented image(s) 306 using one or more graphical indicators. For example, one or more color indicators, shape indicators, pattern indicators, shading indicators, lines, curves, markers, labels, tags, text features, other types of graphical indicators, or a combination thereof may be used to identify the portion(s) of an CFP image that have been identified as a retinal element.

In some embodiments, the CFP segmentation system 304 may include one or more machine learning models (e.g., one or more deep learning models, such as, but not limited to CNNs) that can perform retinal segmentation on the preprocessed image(s) 116 and/or the CF imaging data 300 to generate segmented image data. The CFP segmentation system 304 may further include one or more machine learning models (e.g., one or more deep learning models, such as, but not limited to CNNs) that can extract the retinal data 214 from the segmented image(s) 306 (segmented image data).

The segmented image(s) 306 may include the retinal data 308. In some embodiments, the CFP segmentation system 304 generates the retinal data 308. The retinal data 308 may include values for various retinal features relating to one or more pathological elements of the retina, one or more layers of the retina, or both. The retinal data 308 may include, for example, without limitation, feature data extracted from the segmented image data described above. For example, feature data may be extracted for one or more retinal elements identified in the segmented image data associated with the segmented image(s) 306. This feature data may include values for any number of or combination of features (e.g., quantitative features). These features may include pathology-related features, layer-related volume features, layer-related thickness features, or a combination thereof. Examples of feature data may include DR lesion measurements such as for example a cotton-wool-spot-area to available-field-area ratio, a hard-exudate-area to available-field-area ratio, a hemorrhage area to available-field-area ratio, and a microaneurysm count.

As illustrated in FIG. 3 and when the treatment prediction system 100 uses the CF imaging data 300, the prediction model 106 may be or include the CF model 302 to generate the treatment prediction output 120 based on the segmented image(s) 306 alone or in combination with the medical data 104. In some embodiments, at least some of the input data, which includes the CF imaging data 300 and the medical data 104, may be directly input into the CF model 302. For example, in some embodiments, the medical data 104 and/or the CF imaging data 300 is input into the CF model 302. In some embodiments, the CF model 302 may include a single modal RETFound foundation model or a multi-modal RETFound foundation model.

The CF model 302 uses the segmented image(s) 306 to generate the treatment prediction output 120, which predicts treatment response for a subject with DME. For example, the CF model 302 may provide a predicted 1-year BCVA for the subject associated with the segmented image(s) 306 or provide a prediction for some other future point in time. In other examples, the treatment prediction output 120 generated by the CF model 302 includes a predicted difference between a central subfield thickness (CST) at baseline and a future point in time.

In some embodiments, the CF model 302 generates the treatment prediction output 120 described in FIG. 1 based on the CF imaging data 300. In some embodiments, the CF model 302 generates the treatment prediction output 120 described in FIG. 1 based on CF imaging data 300 from a baseline timepoint and one or more timepoints after the baseline timepoint. That is, the CF imaging data 300 may be associated with a subject at a first point of time and a second point of time that is later than the first point of time.

While FIG. 3 illustrates an image preprocessor 110, the image processor 112, and the CF model 302 as separate components, in some embodiments the CF model 302 is an end-to-end deep learning model configured to use raw CF imaging data 300 and produce the treatment prediction output 120 and/or the treatment output 122.

In some embodiments, the treatment prediction output 120 generated by the CF model 302 is used to generate the treatment output 122 described in FIG. 1.

In some embodiments, the treatment prediction system 100 that includes the CF model 302 predicts treatment response for patients with better accuracy and consistency than expert human graders. The treatment prediction system 100 that includes the CF model 302 provides a technical effect of improving accuracy, reducing the overall computing resources, and/or reducing the time needed to predict a specific subject's predicted treatment response. Further, using the treatment prediction system 100 that includes the CF model 302 may help improve overall treatment management of DME as compared to other methods and systems.

In some embodiments, the treatment prediction system 100 that includes the CF model 302 provides a technical improvement to the field of DME treatment and/or the technical field of predicting a specific subject's predicted treatment response. As noted above, the treatment prediction system 100 that includes the CF model 302 predicts treatment response for patients with better accuracy and consistency than expert human graders and may reduce the overall computing resources and/or time needed to predict treatment response for subjects.

### III. Example Methodologies for Predicting Treatment Response for DME

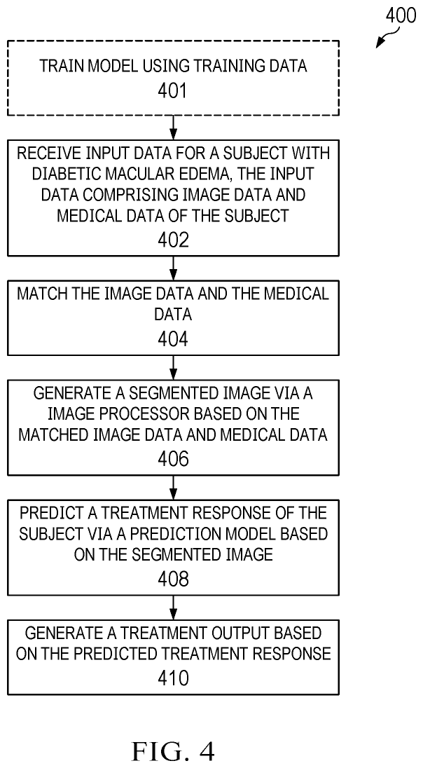

FIG. 4 is a flowchart of a process 400 for predicting treatment response in accordance with one or more embodiments. In one or more embodiments, the process 400 may be implemented using any one of the treatment prediction systems 100 described in FIGS. 1-3. The process 400 includes various steps and may be described with continuing reference to FIGS. 1-3. One or more steps that are not expressly illustrated in FIG. 4 may be included before, after, in between, or as part of the steps of the process 400. In some embodiments, the process 400 may begin with step 402.

The process 400 may optionally include the step 401 of training a model (e.g., a machine learning model or deep learning model). Training the model may include any one of the example trainings described below. The model may be trained to process image data and generate a treatment prediction output and/or a treatment output. The treatment prediction output may be, for example, the treatment prediction output 120 in FIG. 1 and the treatment output may be, for example, the treatment output 122 in FIG. 1.

Step 402 includes receiving input data for a subject with diabetic macular edema, the input data comprising image data and/or medical data of the subject. The input data may be, for example, the image data 102 of FIG. 1, the OCT imaging data 200 of FIG. 2, and/or the CF imaging data of FIG. 3. The medical data may be, for example, the medical data 104 of any one of FIGS. 1-3. The retina may be a retina diagnosed with or suspected of having a retinal disease. The retinal disease may be, for example, age-related macular degeneration (AMD), diabetic macular edema (DME), or some other type of retinal disease. In other embodiments, the retina may be a healthy retina or a retina for which no diagnosis has yet been made.

The step 404 includes matching the image data and the medical data. For example, the medical data and image data may be matched at the subject level or at the eye level. In some embodiments, an image preprocessor matches the image data and the medical data. The image preprocessor may be, for example, the image preprocessor 110 of any one of FIGS. 1-3.

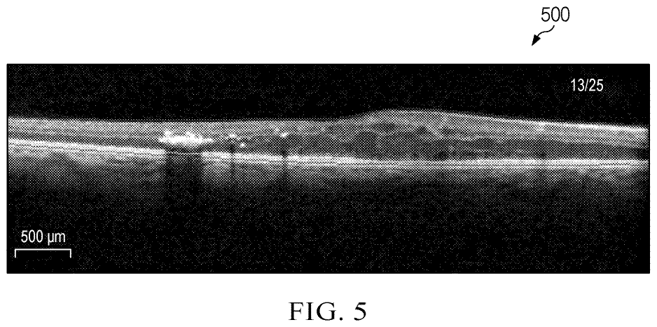

The step 406 includes generating a segmented image via an image processor based on the matched image data and medical data. In some embodiments, the preprocessed image(s) 116 of any one of FIGS. 1-3 comprises the matched image data and medical data. The image processor may include one or more of the image processor 112 in FIG. 1, the OCT image segmentation system 210 in FIG. 2, and/or the CFP segmentation system 304 in FIG. 3. The segmented image may be the processed image(s) 118 in FIG. 1, the segmented image(s) 212 in FIG. 2, and/or the segmented image(s) 306 in FIG. 3. FIG. 5 is an example segmented image 500 of an OCT image.

The step 406 includes predicting a treatment response of the subject via a prediction model based on the segmented image. The prediction model may be prediction model 106 in FIG. 1, the OCT model 216 in FIG. 2, or the CF model 302 in FIG. 3. The predicted treatment response that is predicted may be, for example, the treatment prediction output 120 in FIG. 1.

The step 410 of the process 400 includes generating a treatment output using the predicted treatment response. The treatment output may be, for example, the treatment output 122 of FIG. 1. In one or more embodiments, the treatment output includes administering an appropriate treatment to the patient identified as a subject with a predicted measurement that exceeds a threshold predicted measurement. In some embodiments, the appropriate treatment may include a monoclonal antibody treatment, such as faricimab, or an anti-Vascular Endothelial Growth Factor (anti-VEGF) treatment, such as ranibizumab.

In some embodiments, the process 400 predicts treatment response for patients with better accuracy and consistency than expert human graders. The process 400 provides a technical effect of improving accuracy, reducing the overall computing resources, and/or reducing the time needed to predict a specific subject's predicted treatment response. Further, process 400 may help improve overall treatment management of DME as compared to other methods and systems.

In some embodiments, the process 400 provides a technical improvement to the field of DME treatment and/or the technical field of predicting a specific subject's predicted treatment response. As noted above, the process 400 predicts treatment response for patients with better accuracy and consistency than expert human graders and may reduce the overall computing resources and/or time needed to predict treatment response for subjects. In some embodiments, the process 400 includes a new combination of steps that results in the technical improvement over conventional DRT detection methods.

### IV. Example Training and Validation of the System

### IV.A. Training and Validation of the Machine Learning Models Using OCT Images

### 1. Example Data for Training ML Models Using OCT Images

Various machine learning models were trained and their performance evaluated. Training including using training data obtained from and/or generated based on data obtained from one or more clinical trials. In particular, 416 patients were selected from the 632 patients in the phase 3 YOSEMITE (NCT03622580) and RHINE (NCT03622593) trials who received Faricimab 6.0 mg every 8 weeks. The selected patients were pooled, stratified, and split so that 70% of the patients were assigned to a training set, 15% of the patients were assigned to a test set, and 15% of the patients were assigned to a holdout set. The selected patients were stratified using the following factors: baseline BCVA, baseline CST, baseline HbA1c level, and baseline DRSS. The training set was split into 5 folds for cross-validation (“CV”). In some embodiments, the B-scans were augmented to include affine transformations and color distortions.

The 416 patients selected included those for which baseline OCT images from the Heidelberg Spectralis and example target variables, such as for example the availability of BCVA at week 4 (after 1 injection), week 24 (after 6 monthly loading injections), and 1 year (average over week 48, 52, and 56), were available.

For training of the machine models, the OCT volume scans were segmented using a pretrained segmentation model (e.g., the OCT image segmentation system 210 in FIG. 2). The OCT image segmentation system 210 was pretrained based on annotations made by certified graders. The OCT image segmentation system 210 was trained to automatically segment retinal pathological elements including fluid measurements and retinal layers measurements.

Based on the segmented image(s), retinal feature data was extracted using a feature extraction model (e.g., the OCT image segmentation system 210 in FIG. 2). In some embodiments the retinal feature data is the retinal data 214 in FIG. 2. In some embodiments, the feature extraction model automatically extracted quantitative retinal features. Specifically, these retinal features include volumetric features (e.g., IRF volume and a SRF volume in the center 1 mm and inner subfields) and retinal layers measurements (e.g., mean retinal thickness measured from the ILM to BM). In some embodiments, all features were derived for each individual B-scan of the OCT volume scan and then aggregated over region(s) (e.g., over the ETDRS grid) from volume-wide measurements.

For training of the machine models, clinical data was also received. In some embodiments, the clinical data is or includes at least a portion of the medical data 104 as described in FIG. 2, with the medical data 104 being associated with the 416 selected patients. In some embodiments, the medical data included demographic data for example age and sex, treatment status for example treatment naïve flat, and disease status, such as for example baseline BCVA, baseline CST, baseline HbA1c, baseline DRSS, the presence of SRF, DRIL, ELM disruption, EZ disruption, and ERM according to expert manual image grading.

### 2. Training of Machine Learning Models Using OCT Images

The functional target was set as the BCVA defined by Early Treatment Diabetic Retinopathy Study (ETDRS) letter scores and the target time points were set at week 4 after 1 injection, week 24 after 6 monthly injections, and year 1 (averaged over weeks 48, 52, and 56).

The baseline features were used to predict the treatment outcomes. A set of input baseline features was selected and divided into tiers, such as for example Tier 0 included BCVA; Tier 1 included Tier 0, age, sex, treatment-naïve flag, CST, HbA1c, and DRSS; Tier 2 included Tier 1, the presence of SRF, DRIL, ELM disruption, EZ disruption, and ERM according to expert manual image grading; and Tier A included the mean retinal thickness between ILM and BM, IRF volume, SRF volume in the center 1 mm and inner subfields. Combinations were taken of the Tiers creating combination Tier-0, Tier0A, Tier-1, Tier-1A, Tier-2, and Tier2A.

The various machine learning models trained included a RETFound model, a ResNet model, an Elastic Net model, a random forest model, a support vector machine model, a regression model, an Extreme Gradient Boosting (XGBoost) model. The models were trained, with the hyperparameters of each pair of model and input feature tier being optimized via CV on the development set. In some embodiments, the model hyperparameters were tuned in grid search with 5 fold CV using the training set. In one embodiment, a best performing model and input feature set was selected for evaluation on the holdout set for each target timepoint.

### 3. Machine Learning Model Performance

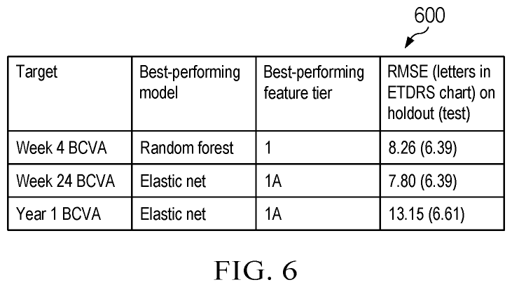

The root mean squared error was used to quantify model performance. FIG. 6 includes a table 600 comparing the statistical results for machine learning models that use image data (e.g., Tier 1) or image data in combination with medical data (e.g., Tier-1A) in accordance with one or more embodiments. As shown in table 600, for the target of Week 4 BCVA, the random forest model using the Tier-1 input feature set performed best. For the target Week 24 BCVA, the elastic net model using Tier-1A input feature set performed best. For the target Year 1 BCVA, the elastic net model using the Tier-1A input feature set performed best.

In some embodiments, the prediction model 106 and/or OCT model 216 is the random forest model using the Tier 1 input feature set and the treatment prediction output 120 is the treatment prediction at “Week 4 BCVA”. In some embodiments, the prediction model 106 and/or OCT model 216 is the Elastic net model using feature Tier-1A input feature set and the treatment prediction output 120 is the treatment prediction at “Week 24 BCVA”. In some embodiments, the prediction model 106 and/or OCT model 216 is the Elastic net model using feature Tier-1A input set and the treatment prediction output 120 is the treatment prediction at “Year 1 BCVA.”

### IV.B. Training and Validation of Deep Learning Models for Predicting Treatment Response for DME Using OCT Images

### 1. Example Data for Training Deep Learning Models Using OCT Images

Various deep learning models were trained and their performance evaluated. Training including using training data obtained from and/or generated based on data obtained from one or more clinical trials. In particular, 772 patients were selected from patients in YOSEMITE (NCT03622580) and RHINE (NCT03622593) trials who received Faricimab every 8 weeks or personalized treatment interval arms. The 772 patients selected included those for which baseline OCT images from the Heidelberg Spectralis were available. The selected patients were pooled, stratified, and split so that 621 of the patients were assigned to a development set and 151 of the patients were assigned to a test set. The selected patients were stratified using the following factors: baseline BCVA, baseline CST, baseline HbA1c level, and baseline DRSS. The training set was split into 5 folds for cross-validation (“CV”). In some embodiments, the B-scans were augmented to include affine transformations and color distortions.

For training of the models, the OCT volume scans were segmented using a pretrained segmentation model (e.g., the OCT image segmentation system 210 in FIG. 2). The OCT image segmentation system 210 was pretrained based on annotations made by certified graders. The OCT image segmentation system 210 was trained to automatically segment retinal pathological elements including fluid measurements and retinal layers measurements.

Based on the segmented image(s), retinal feature data was extracted using a feature extraction model (e.g., the OCT Image segmentation system 210 in FIG. 2). In some embodiments the retinal feature data is the retinal data 214 in FIG. 2. The feature extraction model automatically extracted quantitative retinal features. Specifically, these retinal features include volumetric features (e.g., IRF volume and a SRF volume in the center 1 mm and inner subfields) and retinal layers measurements (e.g., mean retinal thickness measured from the ILM to BM). In some embodiments, all features were derived for each individual B-scan of the OCT volume scan and then aggregated over region(s) (e.g., over the ETDRS grid) form volume-wide measurements.

For training of the models, clinical data was also received. In some embodiments, the clinical data is or includes at least a portion of the medical data 104 as described in FIG. 2, with the medical data 104 being associated with the 772 selected patients. In some embodiments, the medical data included baseline BCVA, baseline CST, baseline HbA1c level, and baseline DRSS; sex, age, diabetes type, and treatment-naïve flag.

### 2. Training of Deep Learning Models

The functional target was set as the BCVA defined by ETDRS letter scores at year 1 (averaged over weeks 48, 52, and 56). The baseline features were used to predict the treatment outcomes.

The various deep learning models trained included a RETFound foundation model and a ResNet model. A single modal RETFound foundation model was trained using OCT images and a multimodal RETFound foundational model was trained using OCT images and the medical data. In some embodiments, the multimodality data included the OCT imaging data 200 and the medical data 104 including baseline BCVA, baseline CST, baseline HbA1c level, baseline DRSS, sex, age, diabetes type, and treatment-naive status. Hyperparameter selection per example model was conducted by choosing the hyperparameter with the lowest mean absolute error across the folds in the development dataset. In some embodiments, the model hyperparameters were tuned in grid search with 5 fold CV using the training set.

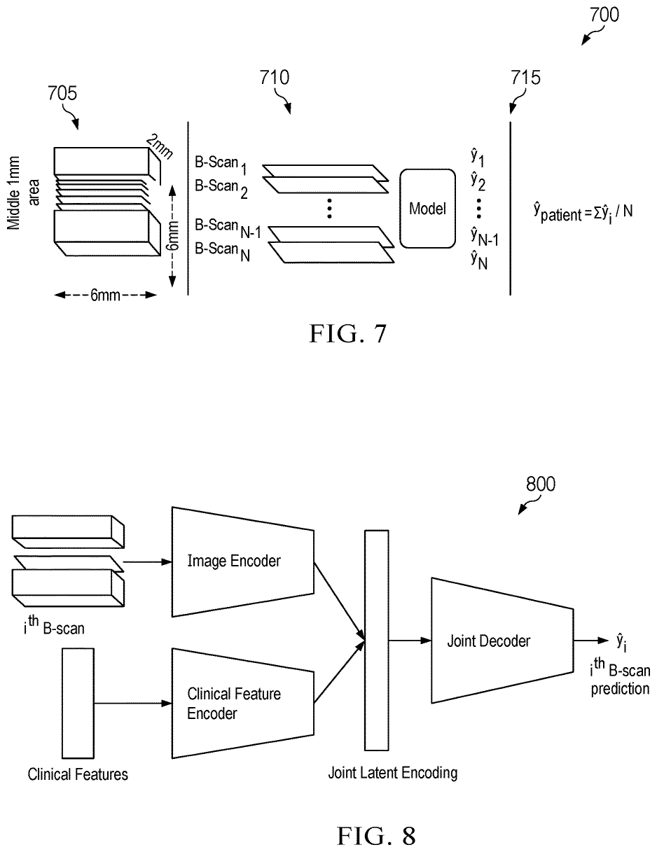

FIG. 7 includes an illustration 700 illustrating an example methodology for making a prediction from an OCT volume. The OCT images were B-scans within the center 1 mm area of the OCT volume. At step 705, the model selects middle B-scans from the OCT volume. At step 710, the model makes a prediction for each B-scan. At step 715, the model then aggregates the predictions of each B-scan for each patient. As such, the patient level prediction when the model is trained to make a prediction from an OCT volume is the average prediction across B-scans. FIG. 8 includes an illustration 800 illustrating an example methodology for a multimodal model to make a prediction. The OCT images were B-scans within the center 1 mm area of the OCT volume. As illustrated, the model selects middle B-scans from the OCT volume. Each middle B-scan from the OCT volume is encoded into an embedding and the clinical features are independently encoded into an embedding. The penultimate layer of the RETFound is used as the image encoder. A dense multilayer perceptron of 2 layers is used as the clinical feature encoder. These embeddings where concatenated and jointly decoded to form a prediction for the sampled B-scan. Finetuning protocols were followed with minor adaptations to the regression task to minimize the MAE loss. With respect to the pretrained ResNet, the pretrained ResNet was fine-tuned and underwent similar hyperparameter tuning and evaluation. For the multimodal ResNet, the clinical data was concatenated to an intermediate representation learned by the model from images and a prediction head was formed by a multilayer perceptron with 1 hidden layer.

### 3. OCT Deep Learning Model Performance

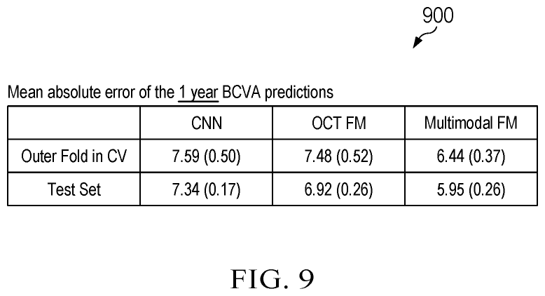

In some embodiments, the mean absolute error of the 1 year BCVA predictions was used to quantify model performance. FIG. 9 includes a table 900 comparing the performance of the various models. As shown in table 900, the CNN (e.g., ResNet) has a MAE of 7.59 for the outer fold in CV and 7.34 in the test set; the OCT FM has a MAE of 7.48 for the outer fold in CV and 6.92 in the test set; and the multimodal FM foundational model has a MAE of 6.44 for the outer fold in CV and 5.95 in the test set.

In some embodiments, the prediction model 106 and/or OCT model 216 is the CNN model, the OCT FM model, and/or the FM foundational model and the treatment prediction output 120 is the treatment prediction at 1 year BCVA.

### IV.C. Training and Validation of Deep Learning Models for Predicting Treatment Response for DME Using CF Images

### 1. Example Data for Training Deep Learning Models Using CF Images

Various deep learning models were trained and their performance evaluated. Training including using training data obtained from and/or generated based on data obtained from one or more clinical trials. In particular, 944 patients were selected from patients in YOSEMITE (NCT03622580) and RHINE (NCT03622593) trials who received Faricimab every 8 weeks or personalized treatment interval arms. The selected patients were pooled, stratified, and split so that 776 of the patients were assigned to a development set and 168 of the patients were assigned to a test set. The selected patients were stratified using baseline BCVA and 1-year BCVA. The training set was split into 5 folds for cross-validation (“CV”). In some embodiments, the CF Images were augmented to include affine transformations and color distortions.

For training of the models, the CF images were processed using a pretrained segmentation model (e.g., the CFP segmentation system 304 in FIG. 3). The CFP segmentation system 304 was trained to automatically identify and/or provide retinal data (e.g., the example retinal data 308 in FIG. 3) for a diabetic retinopathy (DR) lesion measurement, such as for example a CWS area/area ratio, HE area/area ration, HM area/area ratio, and a MA count.

In some embodiments, the CFP images used were 7 standard or 4-wide fields. In some embodiments and for the training of the models, the image preprocessor 110 and/or the image processor 112 comprised an auto-field-classifier that selected field 2 images centered on macula and the image preprocessor 110 and/or the image processor 112 calculated, from the selected field 2 images, the width and height of the minimum-sized image and cropped all images to that size. In some embodiments, the image preprocessor 110 and/or the image processor 112 created a montage of CFP images.

For training of the machine models, medical data was also received. In some embodiments, the clinical data is or includes at least a portion of the medical data 104 as described in FIG. 3, with the medical data 104 being associated with the 944 selected patients. In some embodiments, the medical data included baseline age, gender, treatment naive status, diabetic retinopathy severity scale, hemoglobin A1C, BCVA, and central subfield thickness.

### 2. Training of Deep Learning Models

The functional target was set as the BCVA change from baseline, defined by Early Treatment Diabetic Retinopathy Study (ETDRS) letter scores, to target time points such as for example year 1 (averaged over weeks 48, 52, and 56).

In some embodiments, fine-tuning protocols were followed with minor adaptations to the regression task to minimize the mean squared error (MSE) loss. The various models trained included a multimodal RETFound foundation model. The models were trained, with the hyperparameters of each pair of model and input feature tier being optimized via CV on the development set and then the best-performing model was trained on the entire development set with the best performing hyperparameters for evaluation on the test and holdout set. In some embodiments, the model hyperparameters were tuned in grid search with 5 fold CV using the training set.

### 3. CFP Model Performance

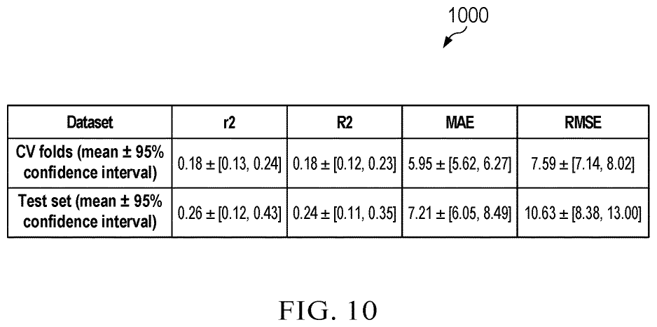

Performance was evaluated by calculating the squared Pearson correlation coefficient (r²), coefficient of determination (R²), mean absolute error (MAE), and root mean squared error (RMSE). The prediction on the test dataset was averaged from the individual predictions from the models trained on the CV datasets and the performance was reported. The multimodal model had a performance of r² of 0.26, R² of 0.24 and MAE of 7.21 letters on the test dataset. FIG. 10 includes a table 1000 that summarizes the performance on the multimodal RETFound foundation model.

### V. Computer-Implemented System

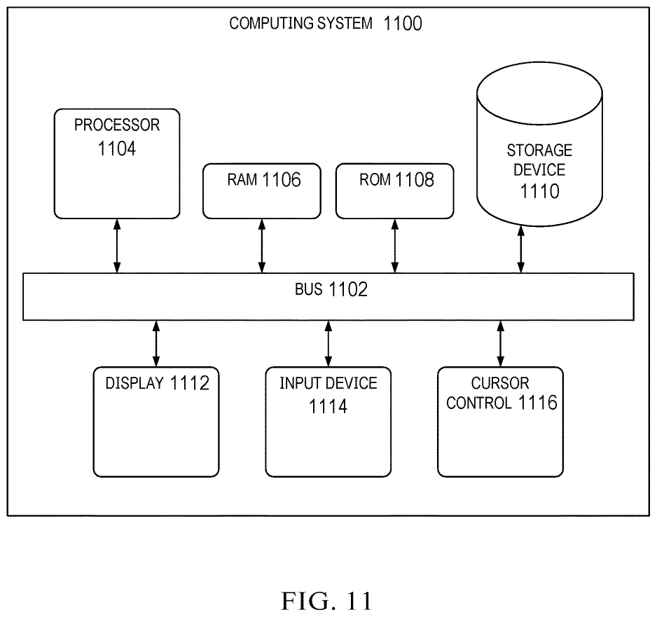

FIG. 11 is a block diagram that illustrates a computer system, in accordance with various embodiments. Computer system 1100 may be one example of an implementation for the computing platform 108 in FIG. 1. In various embodiments of the present teachings, computer system 1100 can include a bus 1102 or other communication mechanism for communicating information, and a processor 1104 coupled with bus 1102 for processing information. In various embodiments, computer system 1100 can also include a memory, which can be a random access memory (RAM) 1106 or other dynamic storage device, coupled to bus 1102 for determining instructions to be executed by processor 1104. Memory also can be used for storing temporary variables or other intermediate information during execution of instructions to be executed by processor 1104. In various embodiments, computer system 1100 can further include a read only memory (ROM) 1108 or other static storage device coupled to bus 1102 for storing static information and instructions for processor 1104. A storage device 1110, such as a magnetic disk or optical disk, can be provided and coupled to bus 1102 for storing information and instructions.

In various embodiments, computer system 1100 can be coupled via bus 1102 to a display 1112, such as a cathode ray tube (CRT) or liquid crystal display (LCD), for displaying information to a computer user. An input device 1114, including alphanumeric and other keys, can be coupled to bus 1102 for communicating information and command selections to processor 1104. Another type of user input device is a cursor control 1116, such as a mouse, a trackball or cursor direction keys for communicating direction information and command selections to processor 1104 and for controlling cursor movement on display 1112. This input device 1114 typically has two degrees of freedom in two axes, a first axis (i.e., x) and a second axis (i.e., y), that allows the device to specify positions in a plane. However, it should be understood that input devices 1114 allowing for 3-dimensional (x, y and z) cursor movement are also contemplated herein.

Consistent with certain implementations of the present teachings, results can be provided by computer system 1100 in response to processor 1104 executing one or more sequences of one or more instructions contained in memory 1106. Such instructions can be read into memory 1106 from another computer-readable medium or computer-readable storage medium, such as storage device 1110. Execution of the sequences of instructions contained in memory 1106 can cause processor 1104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1104 for execution. Such a medium can take many forms, including but not limited to, non-volatile media, volatile media, and transmission media. Examples of non-volatile media can include, but are not limited to, optical, solid state, magnetic disks, such as storage device 1110. Examples of volatile media can include, but are not limited to, dynamic memory, such as memory 1106. Examples of transmission media can include, but are not limited to, coaxial cables, copper wire, and fiber optics, including the wires that comprise bus 1102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1104 of computer system 1100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

In some embodiments, the network 114 may be implemented using a single network or multiple networks in combination. The network 114 may be implemented using any number of wired communications links, wireless communications links, optical communications links, or combination thereof. For example, in various embodiments, the network 114 may include the Internet or one or more intranets, landline networks, wireless networks, and/or other appropriate types of networks. In another example, the network 114 may comprise a wireless telecommunications network (e.g., cellular phone network) adapted to communicate with other communication networks, such as the Internet. In some cases, the network 114 includes at least one of a local area network (LAN), a virtual local area network (VLAN), a wide area network (WAN), a public land mobile network (PLMN), the Internet, or another type of network. The treatment prediction system 100 may each include one or more electronic processors, electronic memories, and other appropriate electronic components for executing instructions such as program code and/or data stored on one or more computer readable mediums to implement the various applications, data, and steps described herein. For example, such instructions may be stored in one or more computer readable media such as memories or data storage devices (e.g., the data storage 126) internal and/or external to various components of the treatment prediction system 100, and/or accessible over the network 114.

It should be appreciated that the methodologies described herein flow charts, diagrams and accompanying disclosure can be implemented using computer system 1100 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1100, whereby processor 1104 would execute the analyses and determinations provided by these engines, subject to instructions provided by any one of, or a combination of, memory components RAM 1106, ROM 1108, and/or storage device 1110 and user input provided via input device 1114.

### VI. Example Definitions and Context

The disclosure is not limited to the example embodiments and applications described herein or to the manner in which the example embodiments and applications operate or are described herein. Moreover, the figures may show simplified or partial views, and the dimensions of elements in the figures may be exaggerated or otherwise not in proportion.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. In various cases, “subject” and “patient” may be used interchangeably herein.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “about” used with respect to numerical values or parameters or characteristics that can be expressed as numerical values means within ten percent of the numerical values. For example, “about 50” means a value in the range from 45 to 55, inclusive.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be used. The item may be a particular object, thing, step, operation, process, or category. In other words, “at least one of” means any combination of items or number of items may be used from the list, but not all of the items in the list may be used. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” may include the practice of using algorithms to parse data, learn from the data, and then make a determination or prediction about something in the world. Machine learning may use algorithms that can learn from data without relying on rules-based programming. Deep learning may be one form of machine learning.

As used herein, an “artificial neural network” or “neural network” (NN) may refer to mathematical algorithms or computational models that mimic an interconnected group of artificial neurons that processes information based on a connectionistic approach to computation. Neural networks, which may also be referred to as neural nets, can employ one or more layers of nonlinear units to predict an output for a received input. Some neural networks may include one or more hidden layers in addition to an output layer. The output of each hidden layer may be used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways; when it is being trained it is in training mode and when it puts what it has learned into practice it is in inference (or prediction) mode. Neural networks may learn through a feedback process (e.g., backpropagation) which allows the network to adjust the weight factors (modifying its behavior) of the individual nodes in the intermediate hidden layers so that the output matches the outputs of the training data. In other words, a neural network may learn by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), a U-Net, a fully convolutional network (FCN), a stacked FCN, a stacked FCN with multi-channel learning, a Squeeze and Excitation embedded neural network, a MobileNet, or another type of neural network.

As used herein, “deep learning” may refer to the use of multi-layered artificial neural networks to automatically learn representations from input data such as images, video, text, etc., without human provided knowledge, to deliver highly accurate predictions in tasks such as object detection/identification, speech recognition, language translation, etc.

### VII. Recitation of Example Embodiments

Embodiment 1: A method, comprising: receiving image data comprising an eye of a subject with diabetic macular edema; forming first image input, using an image processor and the image data, for a prediction model; and generating, by the prediction model comprising a first machine learning model, a treatment response prediction of the subject based on the first image input.

Embodiment 2: The method of embodiment 1, wherein the treatment response prediction comprises a predicted visual acuity.

Embodiment 3: The method of any one of embodiments 1-2, wherein the treatment response prediction comprises a predicted macular thickness at a future point in time.

Embodiment 4: The method of any one of embodiments 1-3, wherein the treatment response prediction comprises a change in macular thickness over a future period of time.

Embodiment 5: The method of any one of embodiments 1-4, wherein the treatment response prediction is a prediction of the treatment response by the subject to a treatment that has not been administered to the subject; and wherein the treatment response prediction comprises a predicted measurement at a future point in time after administering the treatment to the subject.

Embodiment 6: The method of any one of embodiments 1-5, further comprising receiving medical data corresponding to the subject; wherein the prediction model generates the treatment response prediction further based on the medical data.

Embodiment 7: The method of embodiment 6, wherein the medical data comprises at least one of: a baseline diabetic retinopathy severity scale (DRSS) score; a baseline hemoglobin A1C (HbA1C) measurement; a baseline visual acuity; a baseline central subfield thickness; a sex of the subject; an age of the subject; a diabetes type of the subject; a treatment naïve label; or a treatment protocol for the subject.

Embodiment 8: The method of any one of embodiments 6-7, further comprising matching the image data to the medical data; wherein the prediction model generates the treatment response prediction further based on the matched image data and the medical data.

Embodiment 9: The method of any one of embodiments 1-8, wherein the image data comprises an OCT image of the eye of the subject.

Embodiment 10: The method of any one of embodiments 1-9, wherein the first image input for the prediction model comprises a segmented OCT image; wherein the segmented OCT image comprises one of a fluid measurement; a retinal layer measurement; or a measurement map; and wherein generating, by the prediction model, the treatment response prediction of the subject is based on the first image input comprising the one of the fluid measurement; the retinal layer measurement; or the measurement map.

Embodiment 11: The method of any one of embodiments 1-10, wherein the image data comprises an OCT volume; wherein the OCT volume comprises a plurality of B-scans; wherein the first image input for the prediction model comprises a plurality of segmented B-scans; wherein predicting, by the prediction model, the treatment response based on the first image comprises: generating, by the first machine learning model, a prediction for each segmented B-scan in the plurality of segmented B-scans; aggregating, by the first machine learning model, the prediction for each segmented B-scan in the plurality of segmented B-scans; and calculating an average prediction using the aggregated plurality of segmented B-scans; and wherein the treatment response prediction comprises the average prediction.

Embodiment 12: The method of any one of embodiments 1-8, wherein the image data comprises a CFP image of the eye of the subject.

Embodiment 13: The method of embodiment 12, wherein the first image input for the prediction model comprises a segmented CFP image; wherein the segmented CFP image comprises a diabetic retinopathy (DR) lesion measurement or a measurement map; and wherein generating, by the prediction model, the treatment response prediction of the subject is based on the first image input comprising the DR lesion measurement or the measurement map.

Embodiment 14: The method of any one of embodiments 1-13, wherein the image processor comprises a second machine learning model.

Embodiment 15: The method of any one of embodiments 1-14, wherein the first machine learning model comprises at least one of a CNN, vision transformer, or other artificial neural network based model.

Embodiment 16: The method of any one of embodiments 1-15, wherein the first machine learning model comprises at least one of an elastic net model, a random forest model, a support vector machine model, or an extreme gradient boost machine model.

Embodiment 17: The method of any one of embodiments 1-16, wherein the method further comprises: identifying the subject as a subject having a treatment response prediction that exceeds a threshold measurement; and generating a treatment output based on the identification of the subject as a subject having a treatment response prediction that exceeds a threshold measurement.

Embodiment 18: The method of any one of embodiments 1-17, wherein the method further comprises: identifying the subject as a subject having a treatment response prediction that does not exceed a threshold measurement; and generating a treatment output based on the identification of the subject as a subject having a treatment response prediction that does not exceed a threshold measurement.

Embodiment 19: A system comprising: one or more data processors; and a non-transitory computer readable storage medium containing instructions which, when executed on the one or more data processors, cause the one or more data processors to perform one or more of the methods described in one of embodiments 1-18.

Embodiment 20: A computer-program product tangibly embodied in a non-transitory machine-readable storage medium, including instructions configured to cause one or more data processors to perform one or more of the methods described in one of embodiments 1-18.

### VIII. Additional Considerations

The headers and subheaders between sections and subsections of this document are included solely for improving readability and do not imply that features cannot be combined across sections and subsection. Accordingly, sections and subsections do not describe separate embodiments. Any one or more of the embodiments described herein in any section or with respect to any FIG. may be combined with or otherwise integrated with any one or more of the other embodiments described herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure here a computer-program product tangibly embodied in a non-transitory machine-readable storage medium, including instructions configured to cause one or more data processors to perform part or all of one or more methods and/or part or all of one or more processes disclosed herein.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

For example, the flowcharts and block diagrams described above illustrate the architecture, functionality, and/or operation of possible implementations of various method and system embodiments. Each block in the flowcharts or block diagrams may represent a module, a segment, a function, a portion of an operation or step, or a combination thereof. In some alternative implementations of an embodiment, the function or functions noted in the blocks may occur out of the order noted in the figures. For example, in some cases, two blocks shown in succession may be executed substantially concurrently. In other cases, the blocks may be performed in the reverse order. Further, in some cases, one or more blocks may be added to replace or supplement one or more other blocks in a flowchart or block diagram.

Thus, in describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

1. A method, comprising:
receiving image data comprising an eye of a subject with diabetic macular edema;
forming first image input, using an image processor and the image data, for a prediction model; and
generating, by the prediction model comprising a first machine learning model, a treatment response prediction of the subject based on the first image input.

2. The method of claim 1, wherein the treatment response prediction comprises a predicted visual acuity.

3. The method of claim 1, wherein the treatment response prediction comprises a predicted macular thickness at a future point in time.

4. The method of claim 1, wherein the treatment response prediction comprises a change in macular thickness over a future period of time.

5. The method of claim 1,
wherein the treatment response prediction is a prediction of the treatment response by the subject to a treatment that has not been administered to the subject; and
wherein the treatment response prediction comprises a predicted measurement at a future point in time after administering the treatment to the subject.

6. The method of claim 1, further comprising receiving medical data corresponding to the subject;
wherein the prediction model generates the treatment response prediction further based on the medical data.

7. The method of claim 6, wherein the medical data comprises at least one of:
a baseline diabetic retinopathy severity scale (DRSS) score;
a baseline hemoglobin A1C (HbA1C) measurement;
a baseline visual acuity;
a baseline central subfield thickness;
a sex of the subject;
an age of the subject;
a diabetes type of the subject;
a treatment naïve label; or
a treatment protocol for the subject.

8. The method of claim 6, further comprising matching the image data to the medical data;
wherein the prediction model generates the treatment response prediction further based on the matched image data and the medical data.

9. The method of claim 1, wherein the image data comprises an OCT image of the eye of the subject.

10. The method of claim 1,
wherein the first image input for the prediction model comprises a segmented OCT image;
wherein the segmented OCT image comprises one of a fluid measurement; a retinal layer measurement; or a measurement map; and
wherein generating, by the prediction model, the treatment response prediction of the subject is based on the first image input comprising the one of the fluid measurement; the retinal layer measurement; or the measurement map.

11. The method of claim 1,
wherein the image data comprises an OCT volume;
wherein the OCT volume comprises a plurality of B-scans;
wherein the first image input for the prediction model comprises a plurality of segmented B-scans;
wherein predicting, by the prediction model, the treatment response based on the first image comprises:
generating, by the first machine learning model, a prediction for each segmented B-scan in the plurality of segmented B-scans;
aggregating, by the first machine learning model, the prediction for each segmented B-scan in the plurality of segmented B-scans; and
calculating an average prediction using the aggregated plurality of segmented B-scans;
and
wherein the treatment response prediction comprises the average prediction.

12. The method of claim 1, wherein the image data comprises a CFP image of the eye of the subject.

13. The method of claim 12,
wherein the first image input for the prediction model comprises a segmented CFP image;
wherein the segmented CFP image comprises a diabetic retinopathy (DR) lesion measurement or a measurement map; and
wherein generating, by the prediction model, the treatment response prediction of the subject is based on the first image input comprising the DR lesion measurement or the measurement map.

14. The method of claim 1, wherein the image processor comprises a second machine learning model.

15. The method of claim 1, wherein the first machine learning model comprises at least one of a CNN, vision transformer, or other artificial neural network based model.

16. The method of claim 1, wherein the first machine learning model comprises at least one of an elastic net model, a random forest model, a support vector machine model, or an extreme gradient boost machine model.

17. The method of claim 1, wherein the method further comprises:
identifying the subject as a subject having a treatment response prediction that exceeds a threshold measurement; and
generating a treatment output based on the identification of the subject as a subject having a treatment response prediction that exceeds a threshold measurement.

18. The method of claim 1, wherein the method further comprises:
identifying the subject as a subject having a treatment response prediction that does not exceed a threshold measurement; and
generating a treatment output based on the identification of the subject as a subject having a treatment response prediction that does not exceed a threshold measurement.

19. A system comprising:
one or more data processors; and
a non-transitory computer readable storage medium containing instructions which, when executed on the one or more data processors, cause the one or more data processors to:
receive image data comprising an eye of a subject with diabetic macular edema;
form first image input, using an image processor and the image data, for a prediction model; and
generate, by the prediction model comprising a first machine learning model, a treatment response prediction of the subject based on the first image input.

20. A computer-program product tangibly embodied in a non-transitory machine-readable storage medium, including instructions configured to cause one or more data processors to:
receive image data comprising an eye of a subject with diabetic macular edema;
form first image input, using an image processor and the image data, for a prediction model; and
generate, by the prediction model comprising a first machine learning model, a treatment response prediction of the subject based on the first image input.
